(19) United States
(12) Patent Application Publication
(10) Pub. No.: US 2016/0250162 A1
(43) Pub. Date: Sep. 1, 2016

(54) Epinephrine and Benadryl Delivery Method and Apparatus

(71) Applicant: Jeremy Miller, Virginia Beach, VA (US)

(72) Inventor: Jeremy Miller, Virginia Beach, VA (US)

(21) Appl. No.: 14/845,752

(22) Filed: Sep. 4, 2015

Related U.S. Application Data
(60) Provisional application No. 62/121,089, filed on Feb. 26, 2015.

(51) Int. Cl. A61K 31/137; A61K 31/135

(52) U.S. Cl. CPC A61K 31/137; A61K 31/135

(57) ABSTRACT

Embodiments of the present disclosure are generally related to a method and apparatus of delivering medication to treat the symptoms of anaphylactic shock. In particular, certain embodiments deliver dosages epinephrine and Benadryl intramuscularly using an auto-injection device. In certain embodiments, the dosages of epinephrine and Benadryl are delivered in a dosage and timing to increase the efficacy in treating the symptoms of anaphylactic shock.

### CROSS-REFERENCE TO RELATED APPLICATIONS

The present application claims the benefit of U.S. provisional application 62/121,089, filed Feb. 26, 2015.

### FIELD OF INVENTION

Embodiments of the present disclosure are generally related to a method and apparatus of delivering medication to treat the symptoms of anaphylactic shock. In particular, certain embodiments deliver dosages epinephrine and Benadryl intramuscularly using an auto-injection device. In certain embodiments, the dosages of epinephrine and Benadryl are delivered in a dosage and timing to increase the efficacy in treating the symptoms of anaphylactic shock.

### BACKGROUND OF THE INVENTION

For some people, a simple meal or a walk in the park can be deadly. People having severe allergies may take special precautions to ensure their safety during everyday activities. Severe allergic reactions can cause anaphylaxis, which can be deadly. Also called anaphylactic shock, this allergic reaction can occur suddenly, and affect multiple locations of the body simultaneously or near instantaneously. Changes to breathing, skin rashes and swelling, blood pressure, and gastrointestinal effects characterize a severe reaction. The most deadly anaphylactic reaction results in swelling of the patient's airway, which may lead to blockage and suffocation, and possibly a loss of consciousness. Each year in North America, up to 2000 people die from anaphylaxis. Death from anaphylaxis is preventable in many cases. What often distinguishes a fatal outcome from a non-fatal outcome is the rapidity of the appropriate treatment.

Allergic reactions may occur after exposure to foods, insect stings, medications, and other reactive stimuli. A person experiencing anaphylactic shock related to an allergic reaction requires immediate medical care and treatment. Without treatment, the person suffering an allergic reaction can succumb to death in minutes. Food allergies, insect stings, and medications are common triggers for anaphylaxis. Without treatment, the person suffering an allergic reaction can succumb to death within 5 to 30 minutes. The fear of allergic reactions may also impact a person's quality of life. For instance, a person with a known severe food allergy may have difficulty going to restaurants because of a fear of having a reaction. In another instance, a person with an insect sting allergy may be afraid to go hiking or into the woods for fear of being stung. In yet another instance, a person with an unknown reaction to a newly given or prescribed medication may experience a severe reaction such as is common in dental facilities, medical offices, or while taken at home.

Severe allergic reactions can pose a lethal threat to the person suffering the allergy. However, previously available inventions designed to solve the problem caused by a severe allergic reaction do not necessarily incorporate the standard of care used in emergency medicine. In spite of prior art developments in the emergency treatment of anaphylaxis, more than 2000 people die each year due to anaphylaxis in North America. The professional standard of care for anaphylaxis by professional emergency medical responders includes a treatment of intramuscular epinephrine followed by intravenous or intramuscular diphenhydramine (Benadryl). However, anaphylaxis often occurs away from professional care, and, those who are first available to provide treatment are typically bystanders who may or may not be trained in treating someone undergoing anaphylaxis. Although intramuscular epinephrine may be available to these first available persons, typically, only oral diphenhydramine is available, if at all, for use by first available persons. Further, oral medications may be contraindicated for people undergoing anaphylaxis. A solution for standard of care treatment of anaphylaxis that can be given by first available persons is currently unavailable in the prior art or the marketplace.

The prevalent preventative solution to the problem of anaphylaxis is the epinephrine auto-injector apparatus. This apparatus is carried by the person with a known allergy. Epinephrine auto-injectors are sometimes kept in schools or are accessible to daycare providers. The epinephrine auto-injector apparatus contains an epinephrine dose that can reverse the symptoms of anaphylaxis immediately. However, the effects of epinephrine are short lived and wear off relatively quickly. Often, trained emergency responders are not yet on the scene when the epinephrine begins to wear off and the allergic person's life can be in danger once again.

The first symptoms of a severe allergic reaction typically include chest tightness, difficulty breathing, coughing, nausea, abdominal pain, difficulty swallowing, skin redness, itchiness, slurred speech, and confusion among other symptoms. Within a few minutes, the person suffering an allergy can exhibit increasingly severe symptoms. The most severe symptoms may include low blood pressure, weakness, loss of consciousness, change in heart rhythm, rapid pulse, loss of oxygen, blocked airway, hives, severe swelling of the eyes, face, or affected body parts, shock, cardiac arrest, and respiratory arrest. In some cases, within five minutes, anaphylaxis can cause death in the most severe circumstances.

People can come into contact with reaction-causing allergens anywhere. Ideally, a person suffering an allergic reaction would be placed in immediate medical care. However, it is not always possible to get a person suffering an allergic reaction to an emergency department quickly enough to ensure their safety. Allergic reactions often occur away from hospitals in locations such as restaurants, schools, and at home. In worst-case situations, allergic reactions occur when a person is located far from a hospital, such as when in a wilderness environment. In an emergency situation, persons suffering an allergic reaction are often unable to seek help on their own.

Often, the outcome of a person who is suffering an allergic reaction depends upon the care given by one or more other persons who are present when the allergic reaction occurs. Such other persons could include friends, family, and/or strangers who aid the person having the allergic reaction. Often, such other persons lack training in emergency treatment of anaphylaxis or the associated necessary resources. Therefore, most anaphylaxis first aid to a person suffering an allergic reaction is self-administered or is administered by untrained friends and family or untrained good Samaritans.

From a medical perspective, an allergic reaction occurs when an allergen molecule interacts with specific IgE antibodies. This molecular interaction triggers a cascade reaction that includes activation of mast cells, and release of chemical mediators such as histamine, and other additional chemical mediators that trigger the clinical effects of capillary leakage (leading to swelling) that ultimately can lead to clinical shock and asphyxia. Histamine is a substance that plays a major role in many allergic reactions, and has a role in dilating blood vessels and making the vessel walls permeable. During an allergic response, there is a local or systemic release of histamine, typically stored in certain cells such as eosinophils and basophils. Hives and angioedema form when, in response to histamine, blood plasma leaks out of small blood vessels in the skin. Angioedema is a form of hives, but the swelling occurs beneath the skin instead of on the surface. Angioedema is characterized by deep swelling around the eyes and lips and sometimes of the genitals, hands, and feet. It generally lasts longer than hives, but the swelling usually goes away in less than 24 hours. Angioedema of the throat, tongue, or lungs can block airways, causing difficulty breathing, which may become life threatening. Binding of histamine (ligand) to Histamine receptors located on various other cells triggers an immune response. Depending on the number of stimuli, or a person's sensitivity to an allergen, a person can have mild to severe allergic reactions.

One of the most common drugs immediately administered to mitigate the effects of allergic response is epinephrine, or adrenaline. Epinephrine increases vascular tone and cardiac output when injected into a patient's muscle. Epinephrine counteracts many of the most dangerous symptoms of anaphylaxis, including acting as a bronchodilator. Epinephrine is often administered through an intra-muscular injection. Many patients with life-threatening allergies carry an epinephrine auto-injector device so that an untrained bystander can administer the drug in case of emergency. Auto-injectors can also be used by the patient while the patient is still conscious. An injection of epinephrine acts very quickly but the effects do not last very long. Because epinephrine's effect can wear off quickly, persons with known severe allergies will sometimes further carry an emergency kit further having more than one epinephrine auto-injectors, and/or an additional bottle of orally administered diphenhydramine (Benadryl). Orally administered diphenhydramine may come in a liquid form or a pill form.

Diphenhydramine is an anti-histamine which lessens the symptoms of the allergic reaction in a different way than epinephrine. Epinephrine does not directly stop an allergic reaction. Instead, epinephrine lessens the symptoms of anaphylaxis by acting as a bronchodilator, and countering the effects of bronchoconstriction and vasodilation associated with an allergic reaction. On the other hand, diphenhydramine counteracts histamine by antagonizing histamine binding to histamine receptors, such as the H1 receptor. Therefore, administration of orally administered diphenhydramine to a person undergoing anaphylaxis can lessen the overall symptoms of anaphylaxis, where said symptoms typically include vasodilation, exudation, and edema, by directly antagonizing the effects of histamine. In addition, a combination therapy of epinephrine and orally administered diphenhydramine lessens the severe allergy symptoms using two different molecular approaches, which can be more effective than a single drug approach.

However, there are a number of problems associated with prior art methods to administering diphenhydramine for someone undergoing anaphylaxis. In general, oral medication is contraindicated in a person undergoing anaphylaxis due to said person having symptoms of airway swelling, difficulty swallowing, altered mental status, vomiting, and poor compliance. Giving oral medication, such as orally administered diphenhydramine, to an anaphylactic person may be contraindicated due to increased risk of choking An anaphylactic person can have trouble breathing and/or swallowing after onset of symptoms. The difficulty in breathing can be non-life threatening as is the case with wheezing and panic-related hyperventilation, or life threatening as is the case with severe swelling of the mouth and throat. Even mild breathing difficulty increases the risk of choking or aspirating the medication when attempting to swallow. Orally administered diphenhydramine must be ingested, and a person undergoing anaphylaxis has the potential to choke or vomit.

Furthermore, an anaphylactic person can panic during an allergic reaction. Anaphylaxis can cause a panic response in both adults and children. Severe panic can cause a person to be combative, wherein a combative person can fight those who are trying to help. When a person is combative, they are often unwilling or unable to calm down long enough to take an oral medication. Even further, oral medication cannot be given to a person who has lost consciousness due to anaphylaxis.

Additionally, diphenhydramine is not generally the first medication used in the emergency treatment of anaphylaxis because it is relatively slow to act. The timing of action may also depend on the method of diphenhydramine administration. Oral diphenhydramine, when taken in, for example, pill form, can take 30 minutes to over an hour to begin to relieve allergy symptoms, and the onset of action can depend on the amount of food in the stomach. The onset of action for the liquid oral form of diphenhydramine acts faster, and dissolving diphenhydramine tablets act at a similar speed to the liquid form of the drug. In any case of oral intake, vomiting, which can be a common symptom of anaphylaxis, may render the drug less effective.

Therefore, epinephrine, which is faster-acting, is the first choice for immediate treatment of anaphylaxis, while diphenhydramine is used as a second line of defense against the severe allergic reaction. Commonly, an epinephrine auto-injector delivers a dose of 0.3 mg epinephrine (0.3 mL, 1:1000) subcutaneously or intramuscularly, which is the accepted dose in the emergency treatment of allergic reactions. Epinephrine begins to work relatively quickly to reduce the swelling effects associated with anaphylaxis. However, the effect of epinephrine is short lived.

Other inventions of auto-injector apparatuses designed to administer epinephrine in the case of an allergic reaction emergency are described. These devices allow for fast administration of epinephrine by the allergic person himself, or by untrained first available person and trained first responders (see U.S. Pat. No. 7,449,012 by Young et. al. and U.S. Pat. No. 5,295,965 by Wilmot et. al., both incorporated by reference in their entirety). Although these devices are very useful for treatment in an emergency, a single dose of epinephrine, given in association with such auto-injector apparatuses, is only effective for a short amount of time, for example, for about 15 minutes. In many cases, this is not enough time for professional emergency responders to reach the person having an allergic reaction. These problems remain prevalent in many locations in the United States and other countries, especially in places where emergency response times are long. In cases of severe systemic allergic reactions, the people physically nearest the person having the reaction are the ones who are first available to respond even when they have no training in emergency response. There is a need for a first available person to provide treatment necessary to give the best possible treatment to a person experiencing anaphylaxis.

There are a number of descriptions of epinephrine auto-injectors and many improvements on the epinephrine auto-injector. Some improvements are focused on the injecting device itself (U.S. Pat. No. 7,449,012 by Young et. al, and U.S. Pat. No. 5,295,965 by Wilmot et. al., both incorporated by reference in its entirety). Other improvements focus on the composition of the medication, such as stable epinephrine salts (International Publication No. WO 2014/057365 by Gupta et. al., incorporated by reference in its entirety). One improvement in the prior art is a dual injector designed to give two doses of the same medication about 15 minutes apart (or as needed). The art disclosed in U.S. Patent Publication No. 2006/0,129,122 by Wyrick et. al., incorporated by reference in its entirety, injects a single dose of epinephrine and contains a reserve dose available for the likely event that the patient will need a second dose. However, such inventions fail to address the problem that intramuscular injection of epinephrine alone is an insufficient treatment of anaphylaxis.

An injectable anti-histamine using second or third generation, non-sedating, anti- histamines is described in U.S. Patent Publication No. 2012/0010217 by Du, incorporated by reference in its entirety. Second and third generation anti-histamine are known to be less effective against the symptoms of severe allergic reaction than diphenhydramine. In the case of life-threatening conditions, best practices call for an intramuscular or intravenous injection of diphenhydramine. Therefore, prior injectable second and third generation anti-histamines, for example, as described in U.S. Patent Publication No. 2012/0010217 by Du, is not indicated for life threatening allergic reactions.

Two different examples of inventions in the prior art use a single injection of a combination therapy to treat anaphylaxis. In one invention, an anti-depressant is combined in solution with epinephrine to create a therapy designed to be superior to epinephrine alone in the treatment of anaphylaxis (International Publication No. WO 2014/053579 by Schwartz et. al., incorporated by reference in its entirety). In a separate invention designed for less serious allergic reactions (U.S. Pat. No. 6,258,816 by Singh, et. al., incorporated by reference in its entirety), an anti-allergy medication such as Cetrizine, and a non-steroidal anti-inflammatory (NSAID) drug such as Nimesulide are combined into a therapy that has both anti-histamine and anti-inflammatory properties. This combination is given as an injection for non-life threatening allergic reactions.

Professional emergency responders use a combination therapy of injectable epinephrine and injectable diphenhydramine in the treatment of anaphylaxis. Waiting for professional emergency responders to arrive on the scene of an emergency related to an allergic reaction can have a deadly result. Under the best conditions, emergency response times can be too long to help those experiencing severe anaphylaxis. Under rural, remote, or otherwise difficult conditions, emergency response times may be too long for effective treatment of anaphylaxis. A current solution is for a person having a predisposed allergy to carry medication, such as epinephrine injectors, necessary to treat themselves. Given a situation where the person is unable to treat himself, the people nearest the anaphylactic person must respond.

Those first at the scene of an anaphylaxis emergency might be other people in the vicinity of a person undergoing anaphylaxis, for example, other patrons in a restaurant, other people on a camping trip, or a passerby on the street. Sometimes the first available person is a teacher, caretaker or, loved one, or can simply be a good Samaritan who is willing to help the anaphylactic person. It is typically unlikely that the first available person in an anaphylaxis situation is trained in the treatment of anaphylaxis. The problem of an untrained and/or unprepared person giving the standard of care treatment to a person experiencing an anaphylaxis emergency is currently unsolved. Overall, there is a need for a quick, yet more effective intervention to treat those undergoing anaphylaxis. In particular, there is a need for a more efficient delivery system to treat people undergoing anaphylaxis, which can be further performed by the person suffering from anaphylaxis, or by first available persons who may or may not be trained in treating those undergoing anaphylaxis.

### SUMMARY OF THE INVENTION

The present disclosure relates to an emergency response method and apparatus used to administer a therapy. The present disclosure is directed to a method and apparatus to more effectively treat a person undergoing anaphylaxis. Certain embodiments include an injector containing two or more separate medications, primarily epinephrine and diphenhydramine that can be used by an untrained person, mainly for the purpose of treatment for an allergic reaction. Certain embodiments have an auto-injecting device that administers two medications in succession. In the certain embodiments, the first medication administered is epinephrine (adrenaline) and the second medication is diphenhydramine (Benadryl). In certain embodiments, the injector administers both medications intramuscularly. The pairing of an intramuscular epinephrine injector and an intramuscular diphenhydramine injector in certain embodiments allows the quick and effective treatment of a person suffering an allergic reaction by an untrained individual.

### BRIEF DESCRIPTION OF THE DRAWINGS

FIG. 1. Flow diagram disclosure of an embodiment of the present disclosure.

FIG. 2A. An embodiment of the present disclosure comprising epinephrine and diphenhydramine.

FIG. 2B. An embodiment of the present disclosure showing an exposed cannulated needle.

FIG. 3A. A cross-sectional view of an embodiment of the present disclosure.

FIG. 3B. A cross-sectional view of an embodiment of the present disclosure wherein the auto-injector is activated.

FIG. 4. An embodiment of the present disclosure showing the auto-injector apparatus being injected into a user suffering from an allergic reaction.

### DETAILED DESCRIPTION OF CERTAIN EMBODIMENTS

Certain embodiments of the present disclosure are directed to methods and apparatuses that more effectively treat a person undergoing a severe immune response and/or anaphylactic shock. Said methods to effectively treat a person undergoing a severe immune response and/or anaphylaxis further includes a device to administer epinephrine and diphenhydramine intramuscularly. Embodiments of the present disclosure are directed to a combination of epinephrine and diphenhydramine administered intramuscularly with a single device, which increases the effectiveness of the overall treatment, as it bypasses problems associated with a single dose of injectable epinephrine combined with orally administered diphenhydramine. Another benefit of intramuscular administration of a combination of epinephrine and diphenhydramine is that the drug can be given to a patient who is panicking, combative, non-compliant, unconscious, having trouble breathing, vomiting, or otherwise unable to safely take an oral medication. Therefore, certain embodiments of the present disclosure includes an easy to administer, more effective way of treating a person undergoing anaphylaxis even in cases where said person is away from professional emergency medical care.

Certain embodiments of the present disclosure combine the strengths of the standard of care for anaphylactic emergencies currently by both those in the immediate environment and trained medical professionals. When available, those in the immediate environment can use the epinephrine auto-injector to treat a person experiencing a severe allergic reaction. Trained medical professionals are generally encouraged to use injectable epinephrine followed by injectable diphenhydramine to treat a person experiencing a severe allergic reaction. Embodiments of the present disclosure allows anyone treating an anaphylactic person to treat the person with injectable epinephrine followed by injectable diphenhydramine. Currently, prior art that allows for those first administering treatment to administer intramuscular epinephrine followed immediately by intramuscular diphenhydramine in fast succession using one device, is lacking.

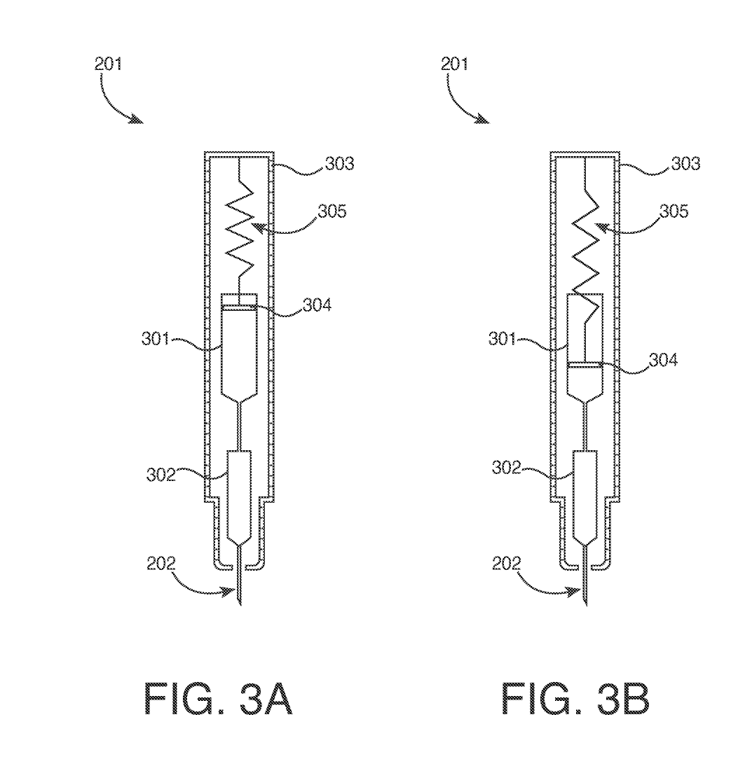

In certain embodiments, the present disclosure utilizes an auto-injector apparatus similar to that described in U.S. Pat. No. 6,270,479 (by Bergens et. al.), which is incorporated by reference in its entirety. In this embodiment auto-injector apparatus 201 is capable of injecting two separate drugs intramuscularly, injected consecutively or simultaneously, through a single injection conduit, as seen in FIG. 3A and FIG. 3B. Certain embodiments of the present disclosure can make use of the best available dual-medication auto-injector to expedite the administration of epinephrine and diphenhydramine to an anaphylactic person provided that the auto-injector is easy to use by an untrained person; the auto-injector apparatus 201 can withstand the physical demands of being transported in a backpack, first aid kit, or other toting system; the auto-injector can be carried for long periods of time in anticipation of use; the auto-injector can be used by the allergic person on himself; and the auto-injector can be used without lengthy preparation. Further, embodiments of the present disclosure may use a pre-existing injection device, such as, for example, the DuoDote® as to inject the epinephrine and the diphenhydramine without taking away from the essence of the invention, as the present invention's use of both epinephrine and the diphenhydramine in a single apparatus solves a number of problems found in the prior art.

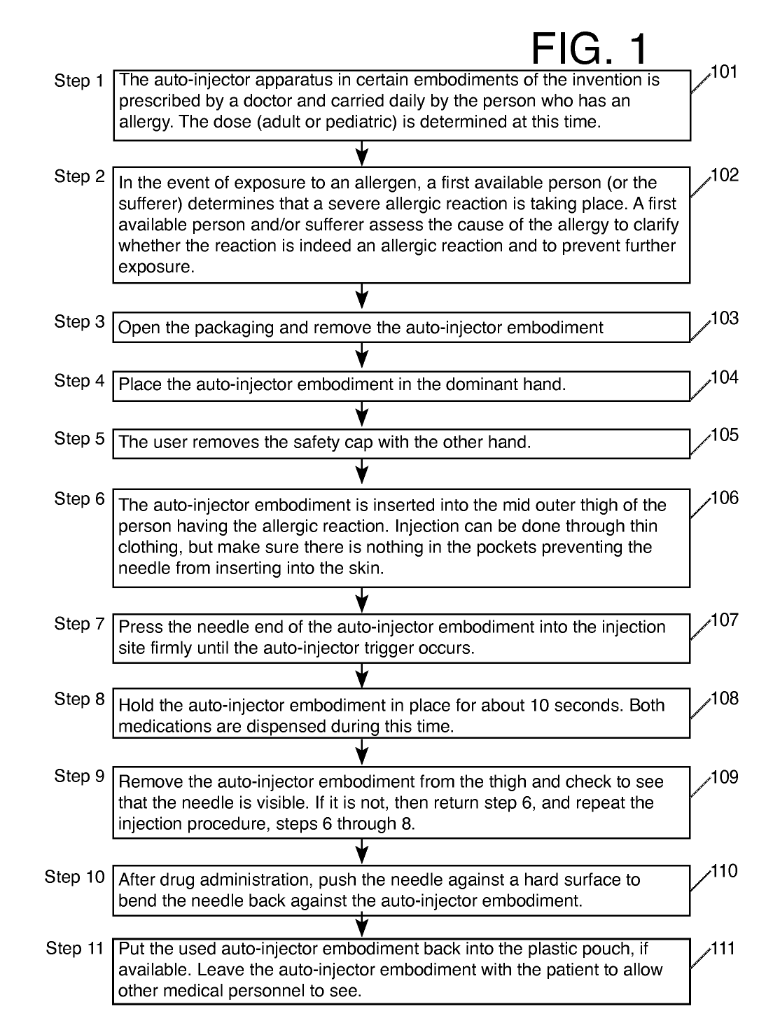

In certain embodiments, the method easily treats a person with anaphylaxis, where such method further includes an auto-injector apparatus to treat a person with anaphylaxis. In certain embodiments, there are a number of steps using an auto-injector apparatus 201. The flow chart, as illustrated in FIG. 1 is a method for treating a person with anaphylaxis. In an exemplary embodiment of the present disclosure, a method is provided for treating a person with anaphylaxis, as follows:
1. (101) The auto-injector apparatus in certain embodiments of the disclosure with 0.3 mg epinephrine and 50 mg diphenhydramine (adult dose) is prescribed by a doctor and carried daily by the person who has an allergy. The dose (adult or pediatric) is determined at this time. In certain embodiments, patients having a weight greater than or equal to 66 lbs (30 kg) receive a dose of 0.3 mg epinephrine and 25 mg diphenhydramine. In certain embodiments, patients having a weight greater than or equal to 66 lbs (30 kg) receive a dose of 0.3 mg epinephrine and 50 mg diphenhydramine. In certain embodiments, a pediatric dose is indicated for those weighing 33 lbs to 66 lbs (15 kg to 30 kg) and contains 0.15 mg epinephrine and 20 mg of diphenhydramine. In yet another embodiment, a pediatric dose contains 0.15 mg epinephrine and 15 mg of diphenhydramine. In yet another embodiment, a pediatric dose contains 0.15 mg epinephrine and 25 mg of diphenhydramine.
2. (102) In the event of exposure to an allergen a first available person (or the sufferer himself) determines that a severe allergic reaction is taking place. Signs of anaphylaxis include more than one of the following symptoms: shortness of breath, throat swelling, hoarseness, difficulty swallowing, hives/rash, swelling in another affected part, vomiting, diarrhea, or other allergy symptoms. The first available person and the sufferer should assess the cause of the allergy to clarify whether the reaction is indeed an allergic reaction and to prevent any more exposure. Immediately seek medical care (i.e. call 911) for the person having an allergic reaction.
3. (103) Open the packaging and remove the auto-injector apparatus 201. Tear open the plastic pouch at any of the notches and remove the auto-injector.
4. (104) Place the auto-injector apparatus 201 in the dominant hand. Firmly grasp the center of the auto-injector with the needle end pointing towards the injection site.
5. (105) The safety cap is removed with the other hand.
6. (106) The auto-injector apparatus needle 202 is inserted into the mid outer thigh of the person having the allergic reaction. This can be done through clothing, but make sure there is nothing in the pockets preventing the needle 202 from inserting into the skin.
7. (107) Press the needle 202 end of the auto-injector apparatus 201 into the injection sight firmly until the auto-injector trigger occurs.
8. (108) Hold the injector in place for about 10 seconds. Both medications are dispensed sequentially during this time.
9. (109) Remove the injector from the thigh and check to see that the needle 202 is visible. If it is not, then be sure to go back to step 6, and repeat the injection procedure from steps 6 to 8. Push the injector harder into the thigh during step 7.
10. (110) After the drug has been administered, push the needle 202 against a hard surface to bend the needle back against the auto-injector.
11. (111) Put the used auto-injector apparatus 201 back into the plastic pouch, if available. Leave the auto-injector with the patient to allow other medical personnel to see.

However, other embodiments of the disclosure are not limited to the foregoing steps, and are meant to be exemplary rather than limiting. An auto-injector apparatus, and further including a delivery means that injects both epinephrine and diphenhydramine is one of the main aspects of embodiments of the present disclosure that solves a number of problems with prior art.

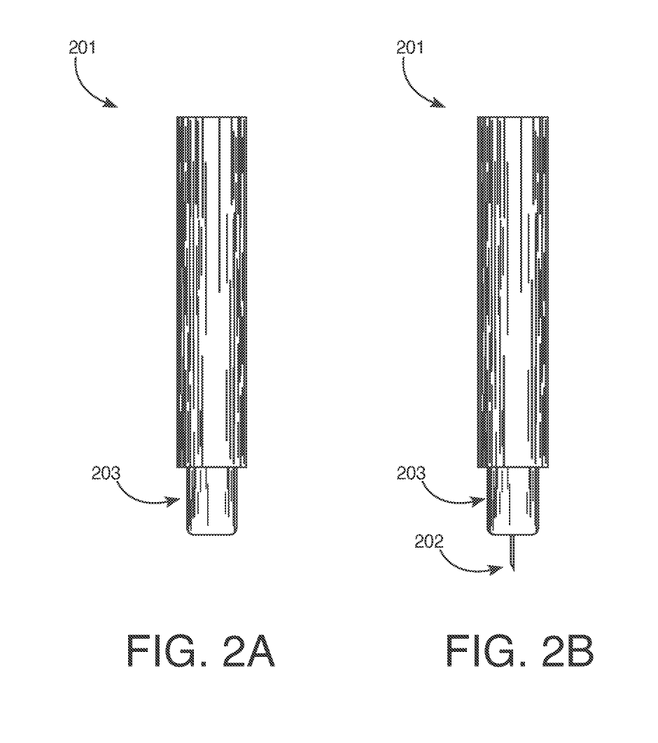

In certain embodiments of the present disclosure, the method to deliver a combination of epinephrine and diphenhydramine is in the form of a dual auto-injector device. Certain embodiments comprise an auto-injector apparatus 201 as illustrated in FIG. 2A. In certain embodiments, the auto-injector apparatus 201 has a form that is easy to handle by a human user, and can be held in one hand. In certain embodiments, the injector is a size that is greater than 2 inches in length, and less than 9 inches in length. However other embodiments may come in a size that is outside the sizes described for the said certain embodiment, as long as a human user can handle said embodiment with ease, for instance, with one hand, or is hand-held. In order for a human user to handle certain embodiments, the auto-injector apparatus 201 further has a form that is a cylindrical shape, as to enable sufficient gripping of the device. However, other embodiments have forms that are shapes other than a cylinder, for example, a rectangular cuboid, a triangular prism, a flattened disc, or other forms that enable a user to handle an auto-injector apparatus with one hand and inject medication intramuscularly.

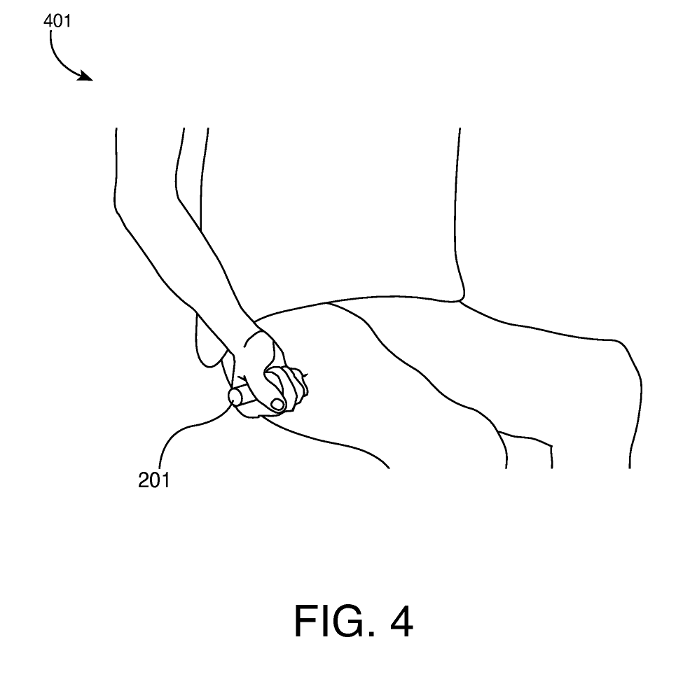

In certain embodiments, the auto-injector apparatus 201 further includes a needle 202, as illustrated in FIG. 2B. In one embodiment, the needle is triggered upon injection of said auto-injector apparatus on a surface, for instance, by pushing the apparatus head 203 of the auto-injector apparatus 201 against the exterior of a body. In an embodiment of the disclosure in use, as illustrated in FIG. 4, a user 401 or a first available person firmly pushes the auto-injector apparatus 201 onto a person undergoing anaphylaxis. Upon pushing of auto-injector apparatus 201 against the exterior of a body, the needle 202 is exposed, enabling the contents of the auto-injector apparatus 201 comprising the epinephrine and diphenhydramine to be injected into a person undergoing anaphylaxis. While certain embodiments have a single needle, other embodiments include an auto-injector apparatus having two needles, wherein each needle leads directly to one container further having a standard care dosing of either epinephrine or diphenhydramine.

In certain embodiments, the present disclosure allows an anaphylactic person to immediately receive the standard of care dosing of epinephrine immediately followed by a dose of diphenhydramine, for instance, 0.3 mg epinephrine 1:1000 followed immediately by 50 mg of diphenhydramine. In certain embodiments, epinephrine and diphenhydramine are administered sequentially and “in rapid succession,” during a single intramuscular injection into the anterolateral aspect of the thigh, through clothing if necessary. In certain embodiments, such rapid succession of epinephrine and diphenhydramine injection occurs within a span of 10 seconds. This dosing is automatically injected when used. In certain embodiments, the epinephrine and diphenhydramine are stored in separate reservoirs within the auto-injector apparatus 201. As illustrated in a cross-sectional view of an embodiment of the disclosure in FIG. 3A, the epinephrine and diphenhydramine are stored in separate containers, as shown in the upper container 301 and the lower container 302. In certain embodiments, the epinephrine is stored in the lower container 302 such that the epinephrine is injected into a person first, followed by the diphenhydramine that is stored in the upper container 301, which is injected second. However, the relative location of the upper container 301 and lower container 302 is not limited to the exemplary drawing in FIG. 3A, as the upper container 301 and lower container 302 may be placed anywhere within and/or outside of the outer casing 303 of the auto- injector apparatus without taking away from the essence of the disclosure. Furthermore, embodiments of the present disclosure further include an apparatus that injects epinephrine into a person prior to injecting diphenhydramine. However, other embodiments include an apparatus that injects diphenhydramine first, or both epinephrine and diphenhydramine simultaneously.

Furthermore, the means to inject the epinephrine and diphenhydramine further includes a resilient member 305, such as, for example, a spring, and a syringe plunger 304, such that a potential force held by the resilient member 305, further fortified by a syringe plunger 304, pushes the contents of the upper container 301 and/or the lower container 302 out through the needle 202, as illustrated in FIG. 3B.

Certain embodiments of the present disclosure take into account that children and smaller people, who require different doses of epinephrine and diphenhydramine. In certain embodiments, a person administers the dose of each drug that is best known to treat smaller people 33 lbs to 66 lbs, which is 0.15 mg of epinephrine and 20 mg of diphenhydramine. The dose is automatically injected when the alternative embodiment of the present disclosure is used with the method described below.

It is the aim, in certain embodiments, to immediately provide medical treatment to a person suffering from an allergic response, by using the beneficial effects of both epinephrine and diphenhydramine in a single injection conduit. Because embodiments utilize liquid diphenhydramine administered intramuscularly, problems and risks associated with administering diphenhydramine through the oral administration, such as choking on the oral administration of diphenhydramine, non-cooperation, unconsciousness and related inability to swallow, and/or vomiting the medication are avoided. Similarly, diphenhydramine typically found in the marketplace or available to the general public is found in a form that can only be administered orally, either in a liquid form or pill form. Further, first available person, or the person suffering from the allergic reaction, who may or may not have prior experience responding to a situation in which a person suffers anaphylaxis, can immediately treat the sufferer with a single and swift injection of both epinephrine and diphenhydramine together.

Moreover, there are a number of advantages to using both epinephrine and diphenhydramine. Because the mechanism of action of diphenhydramine is to antagonize binding of the pro-inflammatory molecule, histamine, to histamine receptors, diphenhydramine works to counter the effects of the localized or systemic allergic by blocking the effects of histamine. Further, the effects of diphenhydramine on the body are much longer than using only epinephrine, which is the typical method of care found in the prior art. Therefore, by combining the effects of epinephrine and diphenhydramine, a person suffering from an allergic reaction is able to get longer lasting, and effective treatment to his or her allergic reaction. In addition, by prolonging the effectiveness of the treatment by using a combination epinephrine and diphenhydramine therapy in the preferred embodiments of the disclosure, more time can be given for an emergency care professional, such as an Emergency Medical Technician who may be on route to care for the person suffering from anaphylaxis, to effectively treat said suffering person, and prevent death, hypoxia, and other consequences related to an anaphylactic shock.

The illustrations of arrangements described herein are intended to provide a general understanding of the structure of various embodiments, and they are not intended to serve as a complete description of all the elements and features of apparatus and systems that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The descriptive labels associated with the numerical references in the figures are intended to merely illustrate embodiments of the invention, and are in no way intended to limit the invention to the scope of the descriptive labels. The present systems, methods, means, and enablement are not limited to the particular systems, and methodologies described, as there can be multiple possible embodiments, which are not expressly illustrated in the present disclosures. It is also to be understood that the terminology used in the description is for the purpose of describing the particular versions or embodiments only, and is not intended to limit the scope of the present application.

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to thosedescribed herein can be used in the practice or testing of embodiments, the preferred methods, and systems are now described. The disclosed embodiments are merely exemplary.

What is claimed is:

1. A method of treating the symptoms of anaphylaxis comprising the steps of:
preparing an auto-injector containing a predetermined dosage of epinephrine and a predetermined dosage of diphenhydramine;
inserting a needle of said auto-injector into a muscle of someone suffering from anaphylaxis;
delivering said dosage of epinephrine and said predetermined dosage of diphenhydramine intramuscularly using said auto-injector apparatus;
and removing said needle of said auto-injector.

2. The method of claim 1 wherein said diphenhydramine dosage is delivered subsequent to said epinephrine dosage.

3. The method of claim 1, wherein said epinephrine dosage ranges from 0.15 mg and 0.3 mg.

4. The method of claim 3 wherein said diphenhydramine dosage ranges from 15 mg to 50 mg.

5. The method of 1 wherein said diphenhydramine dosage ranges from 15 mg to 50 mg.
